(19) United States
(12) Patent Application Publication
(10) Pub. No.: US 2010/0087814 A1
(43) Pub. Date: Apr. 8, 2010

(54) DEVICE FOR CUTTING AND COAGULATING TISSUE

(76) Inventors: Kai Desinger, Berlin (DE); Andre Roggan, Berlin (DE); Thomas Stein, Teltow (DE); Wolfgang Kühne, Schonfliess (DE)

Correspondence Address: WARE FRESSOLA VAN DER SLUYS & ADOLPHSON, LLP, BRADFORD GREEN, BUILDING 5, 755 MAIN STREET, P O BOX 224, MONROE, CT, 06468, US

(21) Appl. No.: 12/303,616

(22) PCT Filed: Jun. 8, 2007
(86) PCT No.: PCT/EP07/05569

§ 371 Date: Apr. 9, 2009

(51) Int. Cl. A61B 18/14

(52) U.S. Cl. 606/45; 606/52

(57) ABSTRACT

The invention concerns an application device for electrosurgical cutting and coagulation of body tissue, wherein the instrument is in the form of a gripping instrument having two gripping arms which are movable relative to each other and which each have at their distal end at least two respective electrodes, namely at least one coagulation electrode and at least one cutting electrode.

The invention concerns an instrument for severing or removing body tissue by means of electrotomy and sclerosing body tissue or staunching hemorrhaging by means of electrocoagulation.

The use of electrosurgical methods for tissue severing or tissue removal (electrotomy) has already been a routine procedure in surgery for decades. Such methods afford the advantage that the tissue is severed in the form of what is referred to as a melt cut in which a spark discharge emanates from the cutting electrode, the spark discharge causing vaporisation of the tissue in the immediate environment of the cutting electrode and immediately closing small capillary vessels so that an almost blood-free tissue cutting procedure is possible. That method is used in all surgical and other medical disciplines for tissue severing or tissue removal.

Electrosurgical sclerosing of tissue or staunching of hemorrhages by means of high frequency current is also a method which is established in surgery and other medical disciplines. The instruments used for that purpose are in the form of gripping instruments such as for example tweezers or forceps. Mounted at the distal ends of the limbs of the gripping instrument are large-area electrodes which are at a different potential (bipolar) or the same potential (monopolar). Both in the monopolar and the bipolar application of the gripping instrument the piece of tissue to be sclerosed or the end of the blood vessel to be closed off is between the electrodes at the distal end of the gripping instrument. In the monopolar procedure the high frequency current flows between those electrodes and a return electrode fixed to the patient at another location. In the bipolar procedure the high frequency current flows between the two electrodes at the distal ends of the limbs of the gripping instrument.

Cutting by means of wire or lancet electrodes disposed at the end of a handle and coagulation with gripping instruments are the high frequency-surgical procedures which are most widespread. In the state of the art at the present time the situation is such that the cutting electrode and the coagulation gripping instrument are two different instruments which, during an operative intervention, often have to be exchanged or operated by two operators.

In the case of operations in the open abdomen area that is not a major problem but in the case of minimally invasive interventions such as laparoscopy or in the case of operations in the oral cavity or the pharynx area that represents a major problem. In laparoscopy a trocar must be fitted for each instrument. If there is a wish to dispense with additional trocars the instruments often have to be changed during the intervention, and that is a time-consuming aspect in laparoscopy.

Interventions in the oral cavity and the pharynx area are carried out only by one operator because of the small angle of view available into that natural body opening. That means that, when changing over from cutting to coagulation, in the presentday state of the art, an instrument change is necessary. The situation is often that, when carrying out a high frequency cut and when severing a major blood vessel, the coagulating property of the coagulated incision edge is not sufficient and in addition the hemorrhaging has to be stopped with a bipolar or monopolar gripping instrument. In such a case a very fast change from the cutting instrument to the coagulating instrument is highly important.

The object of the invention is to provide an application device for electrotomy, which as far as possible avoids the disadvantages of the state of the art.

In accordance with the invention that object is attained by an electrosurgical instrument which is in the form of a gripping instrument and which combines the functions of cutting, coagulating and gripping in one instrument.

For that purpose the instrument is in the form of a gripping instrument having two gripping arms which are movable relative to each other and which each have at their distal end at least two respective electrodes, namely at least one coagulation electrode and at least one cutting electrode.

Preferably a respective cutting electrode is in the form of a wire which is arranged at the outside of a respective distal end of a gripping arm and which thus acts as a cutting wire. In that way the gripping instrument, with the gripping arms closed, can be used in a manner to which the doctor is accustomed, like a monopolar or bipolar cutting instrument of conventional structure.

The coagulation electrodes are preferably in the form of conducting surfaces on mutually facing inside faces at the distal ends of the gripping arms. A mechanical travel limitation for the movement of the gripping arms preferably prevents mutual contact of the conducting surfaces forming the coagulation electrodes, and thus unwanted short-circuiting. The mechanical travel limitation is preferably formed by a projection on an inside surface of a gripping arm, the projection being directed in the direction of the respective other gripping arm.

Preferably at least one of the gripping arms has a suction passage with a suction intake opening in the region of the distal end of the gripping arm, which is to be connected to a suction removal system and is adapted to remove by suction fluid in the region of the distal end of the gripping arm. The suction passage serves to suck away blood or other body fluid which collects at the location of the intervention.

In addition preferably at least one of the gripping arms has a flushing passage which opens in an open end in the proximity of the distal end of the gripping arm and is so adapted that it is to be connected with its proximal end to a source for flushing fluid and flushing fluid can issue by way of its open mouth opening at the distal end. The location of intervention can be flushed with flushing fluid by means of such a flushing passage.

The cutting electrodes are preferably connected in mutually parallel relationship. In addition the application device preferably has a separate neutral electrode which is to be connected to another pole of a cutting current generator, from the two cutting electrodes. In that way the application device operates as a monopolar cutting instrument.

The invention is described in greater detail hereinafter by means of embodiments by way of example and with reference to the accompanying drawings in which:

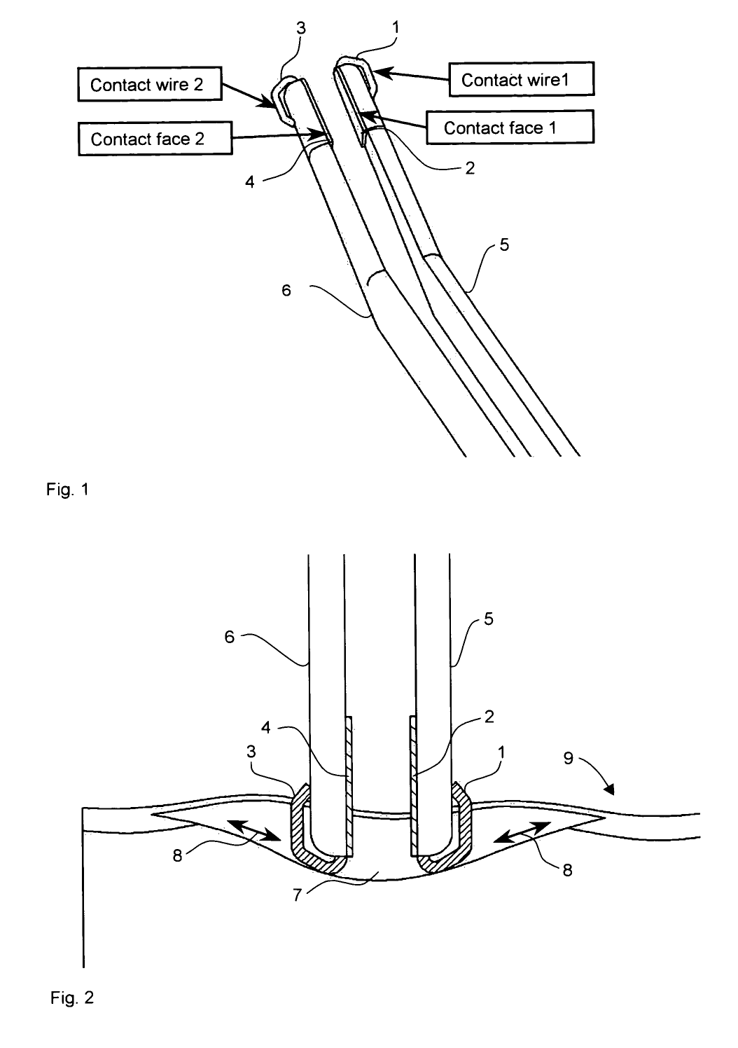

FIG. 1 shows an electrosurgical instrument according to the invention similar to bipolar or monopolar tweezers or forceps, wherein disposed at the insides of the distal ends of the limbs thereof are the large-area electrodes for tissue coagulation (contact faces 1 and 2) and mounted at the outsides of the distal ends of the limbs of which are cutting wires (contact wires 1 and 2);

FIG. 2 shows a diagrammatic view in section on an enlarged scale by way of example of instrument guidance in the electrosurgical severing (electrotomy) of organic tissue by means of the cutting wires mounted on the limb at the outside (contact wires 1 and 2);

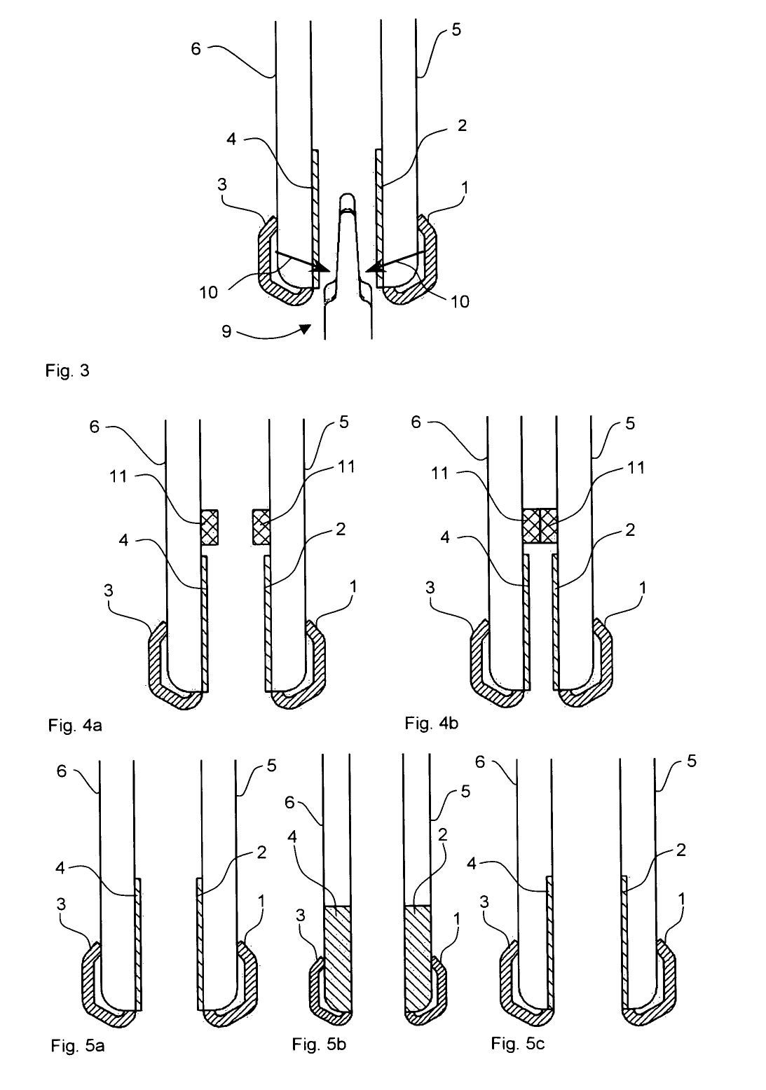

FIG. 3 shows a diagrammatic view in section on an enlarged scale by way of example of an instrument guidance in high frequency coagulation (electrocoagulation) of organic tissue by means of the contact faces on the limbs at the inside thereof (contact faces 1 and 2);

FIGS. 4a and 4b show a diagrammatic view in section on an enlarged scale by way of example of a variant with a short-circuit protection which provides that even when the limbs are closed a minimal gap still remains between the contact faces (contact faces 1 and 2) and thus prevents them from being able to touch each other,

FIGS. 5a, 5b and 5c show diagrammatic views in section on an enlarged scale by way of example of various variants with differently mounted contact faces or integrated contact faces or let-in contact faces,

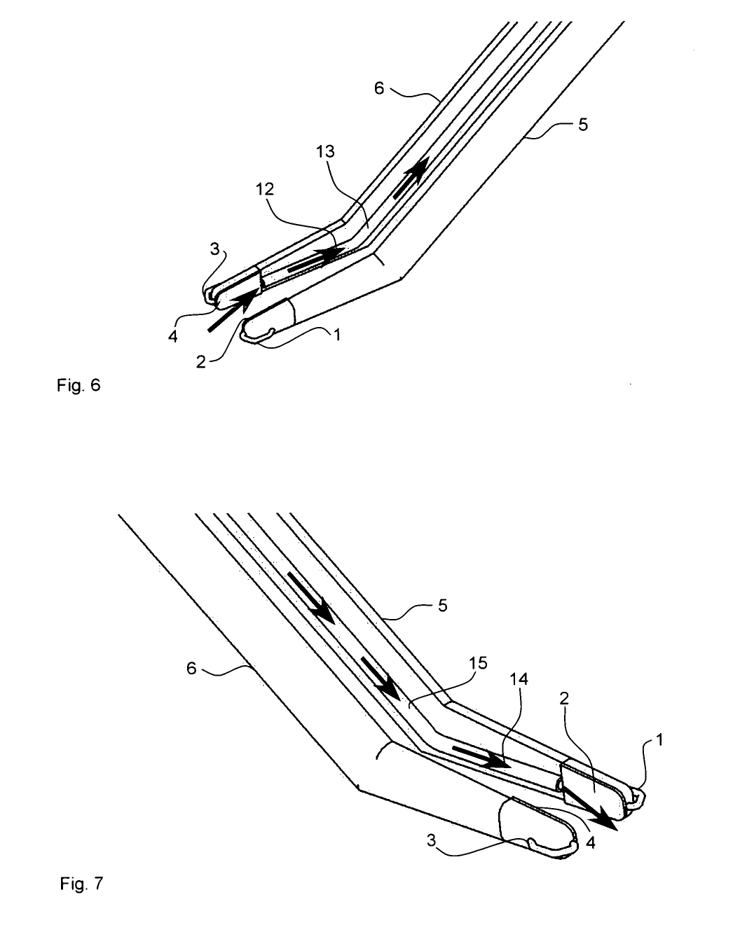

FIG. 6 shows a variant with a suction removal passage which in the immediate working region of the instrument sucks away and transports away fluids such as for example blood,

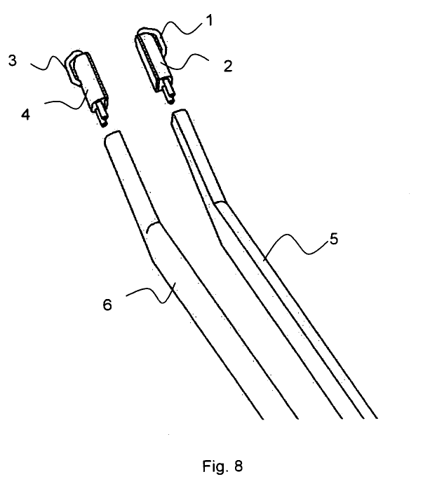

FIG. 7 shows a variant having a flushing passage through which the tissue in the working region can be moistened with fluids such as for example sterile physiological saline solution to prevent the tissue from drying out,

FIG. 8 shows a variant with interchangeable cutting and coagulation electrodes,

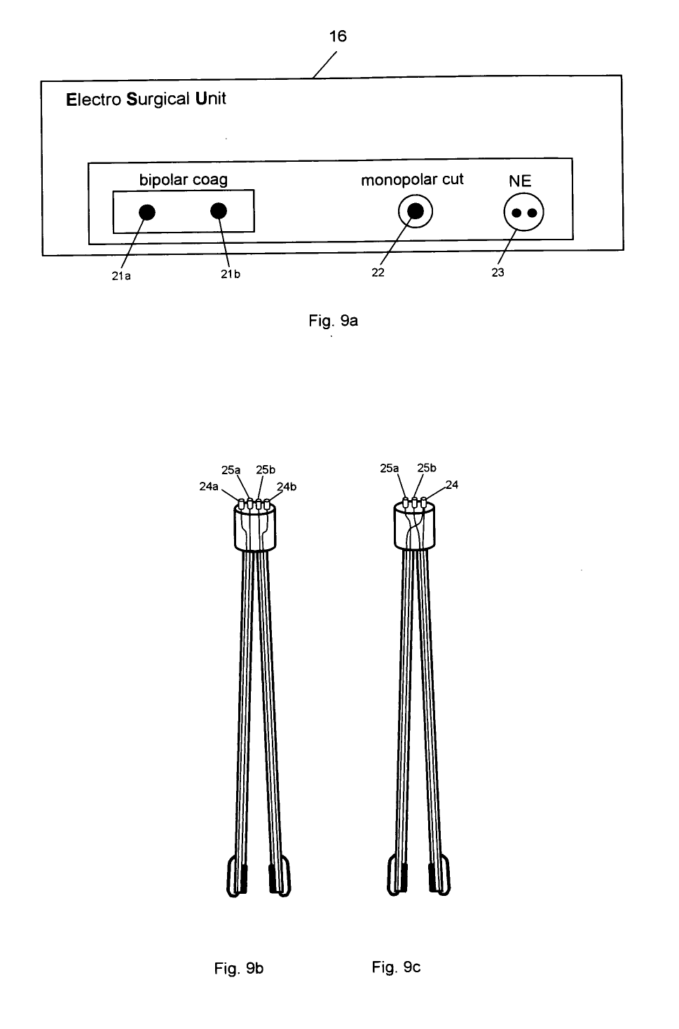

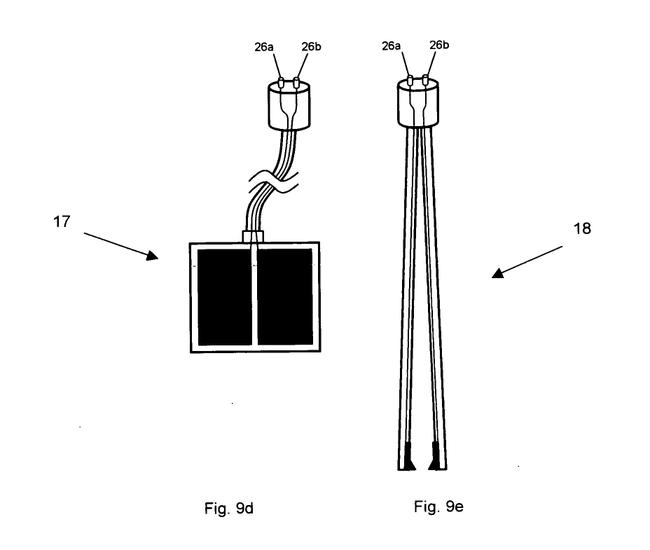

FIGS. 9a through 9e show a high frequency surgery generator (9a) and various application devices to be connected thereto, including the circuitry of the respective electrodes,

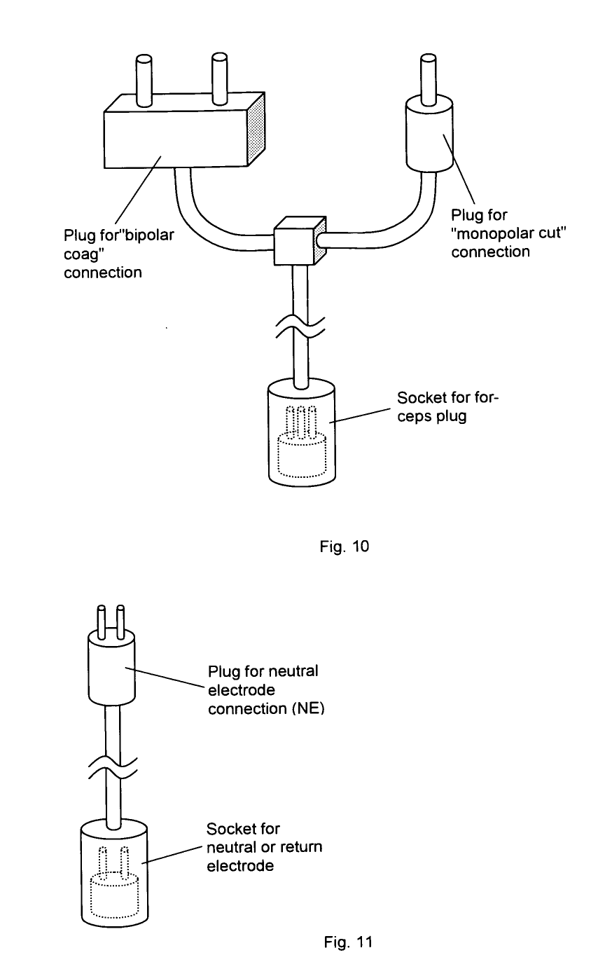

FIG. 10 shows a connecting cable for connecting the electrosurgical instrument according to the invention to the high frequency generator of FIG. 9, and

FIG. 11 shows a connecting cable for connecting a neutral electrode to the high frequency generator of FIG. 9.

FIG. 1 shows a surgical instrument according to the invention which is of a similar structure to bipolar or monopolar tweezers or forceps and which at insides of distal ends of limbs 5 and 6 forming gripping arms, has large-area electrodes 2 and 4 for tissue coagulation, which are also identified as contact faces 1 and 2 in FIG. 1. Mounted to outsides of the distal ends of the limbs 5 and 6 are cutting wires 1, 3 which are also referred to as contact wires 1 and 2 in FIG. 1.

That arrangement thus affords a surgical instrument with a total of four electrodes (two small-area wire electrodes 1 and 3 and two large-area electrodes 2 and 4). The following circuitry configurations/current paths can be envisaged for those four electrodes 1 through 4, as are also graphically shown in FIGS. 9a through 9e:

Cutting Operation:

S1) contact wire 1 to contact wire 2

S2) contact wire 1 to contact face 1

S3) contact wire 1 to contact face 2

S4) contact wire 1 to contact faces 1 and 2

S5) contact wire 2 to contact face 2

S6) contact wire 2 to contact face 1

S7) contact wire 2 to contact faces 2 and 1

S8) contact wires 1 and 2 to contact face 1

S9) contact wires 1 and 2 to contact face 2

S10) contact wires 1 and 2 to contact faces 1 and 2

S11) contact wire 1 to separate return electrode

S12) contact wire 2 to separate return electrode

S13) contact wires 1 and 2 to separate return electrode.

Coagulation Operation:

K1) contact face 1 to contact face 2

K2) contact faces 1 and 2 to separate return electrode.

The electrosurgical instrument (also referred to as the application device) forms a combination instrument provided with a connection which permits an electrical connection to be made to a plurality of outputs of a high frequency surgery generator 16 (Electro Surgical Unit; see FIG. 9a). The high frequency surgery generator 16 has either at least a cutting output 22 and a coagulation output 21a and 21b, or a combination output which provides both a cutting voltage and also a coagulation voltage. When the combination instrument is connected to a conventional high frequency surgery generator having outputs for monopolar cutting, monopolar coagulation, bipolar cutting and bipolar coagulation, it is possible to envisage inter alia the following connecting variants:

output for monopolar cutting for: S11 through S13

output for monopolar coagulation for: K2

output for bipolar cutting for: S1 through S10

output for bipolar coagulation for: K1.

The high frequency surgery generator 16 of FIG. 9a has a connection 21a and 21b for bipolar coagulation (bipolar coag) and a connection 22 for monopolar cutting (monopolar cut) and therefore allows the circuitry variants K1 and S11 through S13 when it is operated with a surgical instrument as shown in FIG. 9b. In that surgical instrument of FIG. 9b, provided at the proximal end of the instrument are a total of four connecting contacts 24a, 24b, 25a and 25b which allow each of the electrodes 1 through 4 to be individually connected to a high frequency surgery generator. The high frequency surgery generator of FIG. 9a in conjunction with a surgical instrument as shown in FIG. 9c allows the circuitry variants K1 and S13. In the surgical instrument of FIG. 9c both cutting wires 1 and 3 are connected to a common connecting contact 24 at the proximal end of the instrument and therefore connected in parallel.

In addition the high frequency surgery generator 16 has a connection 23 for the connection of a neutral electrode (see FIG. 9d or 9e) or a connecting cable for such a neutral electrode (see FIG. 11).

FIGS. 9d and 9e respectively show in particular neutral electrodes suited for the monopolar tissue cutting operation, more specifically FIG. 9d showing a surface electrode 17 as the neutral electrode and FIG. 9e showing a neutral electrode in the form of tweezers 18.

FIG. 2 shows a cutting procedure by way of example, in which, by the guidance movement 8, the cutting wires 1 and 3 on the instrument at the outside thereof are in slight contact with the tissue 9 along the cut edge 7 of the cutting procedure.

FIG. 3 shows a coagulation procedure by way of example, in which the tissue 9 to be coagulated is gripped under a light pressure in the direction 10 between the contact faces 2 and 4 which are disposed inwardly on the instrument.

In bipolar coagulation with a forceps-like tool there is then in principle the risk of a short-circuit if, in the bipolar mode of operation, two electrodes such as for example the cutting wires or the large-area electrodes at the insides of gripping arms touch each other. A short-circuit occurs for example when direct metallic contact between the two contact faces 2 and 4 occurs and the current thus no longer flows by way of the tissue 9. In that case tissue coagulation no longer occurs.

FIGS. 4a and 4b show a preferred variant in which the instrument is provided with a short-circuit protection 11. That short-circuit protection 11 provides a mechanical travel limiting effect and provides that, in the closed condition of the instrument, a minimal gap still remains between the two contact faces 2 and 4 and thus direct metallic contact between the two contact faces 2 and 4 does not take place. The mechanical travel limiting means can be arranged both directly beside the contact faces 2 and 4 and also at any other location on the movable limbs 5 and 6.

FIGS. 5a through 5c show various variants of the arrangement of the contact faces. In FIG. 5a the contact faces 2 and 4 are fixed on the distal ends of the limbs 5 and 6, the limbs comprising a non-conducting material (for example PEEK). In FIG. 5b the instrument comprises solid conductive material (for example surgical steel, V2A), wherein except for the contact faces 2 and 4 the instrument is covered with an insulator (for example of polyacryl, PA). In FIG. 5c the contact faces 2 and 4 are fitted into the distal ends of the limbs 5 and 6, the limbs comprising a non-conducting material (for example polyetheretherketone, PEEK).

In the case of open surgical operations, tissue injuries mean that fluids and secretions specific to the body can be liberated, which then collect at the location to be operated upon and thus have an interfering effect on the operating procedure or OP. In accordance with a conventional method, in such a situation a suction removal instrument is guided to the location to be operated on, in addition to the actual surgical instrument, and that suction removal instrument on the one hand takes up space at the location to be operated on and impedes the OP while on the other hand it also has to be guided separately, possibly by a further person. Under some circumstances the OP even has to be interrupted for sucking away the fluids, as there is not sufficient space for suction removal and OP instruments to be used simultaneously at the OP location.

FIG. 6 shows a preferred variant in which the instrument is provided with a suction removal passage 13 which makes a separate suction removal instrument redundant. The suction removal passage 13 has an open mouth opening as the suction intake opening in the region of the distal ends of the limbs 5 and 6 so that the unwanted fluids which cause a nuisance are sucked away directly in the working region. That is effected in parallel with the normal surgical procedure. The suction removal passage 13 transports the fluid 12 from the suction intake opening along one of the two limbs 5 and 6 to an open proximal end of the suction passage in a region of the instrument, that is remote from the distal end. At its proximal open end the suction removal passage 13 is to be connected to a suction removal system which is usual in the OP.

In electrocoagulation the tissue can dry out during the treatment. The consequence is that the transfer resistance from the tissue to the electrosurgical instruments and also the resistance of the tissue itself rise with increasing drying-out and the procedure no longer operates satisfactorily. At that moment the procedure has to be interrupted to wet the tissue with an ionising, body-compatible fluid (for example sterile physiological saline solution).

FIG. 7 shows a preferred variant in which the instrument is provided with an integrated moistening system. That moistening system includes a flushing passage 15 with an open mouth opening as the outlet opening in the region of the distal ends of the limbs 5 and 6 so that ionising fluid 14 can be metered directly into the working region. That can thus take place in parallel relationship with the normal operating procedure. The flushing passage 15 for the fluid 14 extends along one of the two limbs 5 and 6 and, in the region of the instrument, that is remote from the distal end, is to be connected to a metering system for such fluids, which is usual in the OP.

FIG. 8 shows that the distal ends of the limbs 5 and 6, which include the cutting wires 1 and 3 and the large-area electrodes 2 and 4, can be designed to be interchangeable so that, after exchange of the distal ends, that allows re-use of the rest of the instrument after an operation.

### Use of the Invention for Tonsillectomy:

It is specifically in tonsillectomy that the combination of high frequency cutting and high frequency coagulation in one instrument affords enormous advantages. By means of a plurality of HF incisions by the cutting wires 1 or 3 the tonsil is slowly released from its capsule. In that procedure the operator advances slowly in depth, around the tonsil, between the tonsil and the tonsil capsule. During the cutting operation the tonsil is pulled with tweezers in a median direction out of the tonsil bed. The pincers can at the same time perform the function of a return electrode (see FIG. 9e). In that case small hemorrhages from capillary vessels are stopped immediately by the coagulating action of the HF cut on the incision edges. After the tonsil is released from the capsule the tonsil is removed at the inferior pole, also by an HF cut. When severing larger vessels, in particular arteries, the coagulating action of the HF cut is no longer sufficient to stop hemorrhaging. In that case the end of the hemorrhaging vessel is taken between the contact faces 2 and 4, compressed and an HF current applied. That provides that the opening of the vessel is heat-sealed and the hemorrhaging is stopped. The transition from cutting with the contact wires 1 and 3 to coagulation with the contact faces 2 and 4 can be effected when using the invention without a change of instrument. In that way hemorrhages which occur can be treated more quickly and thus more effectively.

1. An application device for electrosurgical cutting and coagulation of body tissue,
which is in the form of a gripping instrument having two gripping arms (5, 6) which are movable relative to each other, wherein the distal ends of the gripping arms (5, 6) respectively have at least two electrodes, namely at least one coagulation electrode (2; 4) arranged at mutually facing inside faces of the gripping arms (5, 6) and at least one cutting electrode (1; 3), characterised in that the cutting electrode (1, 3) is mounted at the outside of the gripping arm spaced from the inside faces and extends angled from the distal end to a side face of the gripping arm.

2. An application device as set forth in claim 1 characterised in that the respective cutting electrodes (1, 3) are in the form of a wire arranged at the outside of a respective distal end of a gripping arm (5; 6).

3. An application device as set forth in claim 1 characterised in that the coagulation electrodes (2, 4) are in the form of conducting surfaces on mutually facing inside faces at the distal ends of the gripping arms (5, 6).

4. An application device as set forth in claim 3 characterised in that the gripping instrument has a mechanical travel limiting means (11) for the movement of the gripping arms (5, 6), which is so designed that it prevents mutual contacting of the conducting surfaces forming the coagulation electrodes at the distal ends of the gripping arms (5, 6).

5. An application device as set forth in claim 4 characterised in that the mechanical travel limiting means (11) is formed by a projection on an inside face of a gripping arm (6; 5), the projection being directed in the direction of the respective other gripping arm (5; 6).

6. An application device as set forth in claim 1 characterised in that at least one of the gripping arms (5, 6) has a suction passage (13) with an open mouth opening in the region of the distal end of the gripping arm (5; 6), which is to be connected to a suction removal system and is adapted to suck away fluid in the region of the distal end of the gripping arm (5; 6).

7. An application device as set forth in claim 6 characterised in that at least one of the gripping arms (5, 6) has a flushing passage (15) which opens in an open end in the proximity of the distal end of the gripping arm (5; 6) and is so designed that it is to be connected with its proximal end to a source for flushing fluid and flushing fluid can issue by way of its open mouth opening at the distal end.

8. An application device as set forth in claim 1 characterised in that the cutting electrodes are connected in parallel.

9. An application device as set forth in claim 8 characterised by a separate neutral electrode (17; 18) which is to be connected to another pole of a high frequency generator (16), from the two parallel-connected cutting electrodes (1, 3).

10. An application device as set forth in claim 9 characterised in that the separate neutral electrode is in the form of electrosurgical tweezers (18).

11. An application device as set forth in claim 2 characterised in that at least one of the gripping arms (5, 6) has a suction passage (13) with an open mouth opening in the region of the distal end of the gripping arm (5; 6), which is to be connected to a suction removal system and is adapted to suck away fluid in the region of the distal end of the gripping arm (5; 6).

12. An application device as set forth in claim 3 characterised in that at least one of the gripping arms (5, 6) has a suction passage (13) with an open mouth opening in the region of the distal end of the gripping arm (5; 6), which is to be connected to a suction removal system and is adapted to suck away fluid in the region of the distal end of the gripping arm (5; 6).

13. An application device as set forth in claim 4 characterised in that at least one of the gripping arms (5, 6) has a suction passage (13) with an open mouth opening in the region of the distal end of the gripping arm (5; 6), which is to be connected to a suction removal system and is adapted to suck away fluid in the region of the distal end of the gripping arm (5; 6).

14. An application device as set forth in claim 5 characterised in that at least one of the gripping arms (5, 6) has a suction passage (13) with an open mouth opening in the region of the distal end of the gripping arm (5; 6), which is to be connected to a suction removal system and is adapted to suck away fluid in the region of the distal end of the gripping arm (5; 6).
